(19) United States
(12) Patent Application Publication
(10) Pub. No.: US 2024/0408116 A1
(43) Pub. Date: Dec. 12, 2024

(54) COMPOSITIONS FOR ENHANCING SEXUAL PLEASURE WITH REDUCED PHARMACEUTICAL LOAD OF PDE5 INHIBITOR

(71) Applicant: ILYLT, LLC, Murray, UT (US)

(72) Inventor: Dennis M. Jenn, South Jordan, UT (US)

(21) Appl. No.: 18/737,019

(22) Filed: Jun. 7, 2024

Related U.S. Application Data
(60) Provisional application No. 63/471,890, filed on Jun. 8, 2023.

(51) Int. Cl. A61K 31/00; A61K 31/198; A61K 31/375; A61K 31/4985; A61K 31/506; A61K 31/519; A61K 31/53; A61K 36/16; A61K 36/21; A61K 36/258; A61K 36/296; A61K 36/48; A61K 36/74; A61K 36/88; A61K 36/8962; A61P 15/00

(52) U.S. Cl. CPC A61K 31/658; A61K 31/198; A61K 31/375; A61K 31/4985; A61K 31/506; A61K 31/519; A61K 31/53; A61K 36/16; A61K 36/21; A61K 36/258; A61K 36/296; A61K 36/48; A61K 36/74; A61K 36/88; A61K 36/8962; A61P 15/00

(57) ABSTRACT

A composition for increasing sexual pleasure in an adult human includes:
a dosage form selected from capsules, tablets, oral drops, infused edibles, infused food preparations, infused drink preparations, massage oils, lotions, gels, creams, lubricants, patches, and suppositories, the dosage form containing:
a quantity of a PDE5 inhibitor that is no greater than 50% of a full prescription quantity or dose of the PDE5 inhibitor for an adult male; and
a quantity of a cannabinoid component sufficient to amplify the effect of the PDE5 inhibitor to at least 80% of the effect of the full prescription quantity or dose for an adult male,
wherein the PDE5 inhibitor and the cannabinoid component are effective in increasing sexual pleasure in the adult human in need thereof, including at least one of increased blood flow to erectile tissue, increased sexual desire, increased sexual arousal, increased sexual sensitivity, or increased strength of orgasm.

### CROSS REFERENCE TO RELATED APPLICATION

This Application claims the benefit of U.S. Provisional Application No. 63/471,890, filed Jun. 8, 2023, which is incorporated by reference in its entirety.

### BACKGROUND OF THE INVENTION

### 1. Field of the Invention

The invention is in the field of compositions, particularly for sexual enhancement in men, women, the disabled, and the aged, which have reduced pharmaceutical load.

### 2. Relevant Technology

There are a variety of health issues that can impact the ability or desire to engage in intimate sexual relations, which form a healthy part of adult relationships. These include sexual dysfunction in men and women and a loss of sensitivity and pleasure. The inability to perform and/or lack of desire to engage in sexual relations can detrimentally impact a relationship and can lead to divorce, breakup, or long-term boredom. It can lead to loss of self-esteem or even mental illness.

When a man is stressed, anxious or insecure, his ability to perform can be inhibited physically (temporary erectile dysfunction). Older or sick men can suffer chronic erectile dysfunction (“ED”), which can be completely incapacitating relative to ability to perform. Particularly as men age and/or if suffering from chronic illness, they can experience lack of threshold desire, loss of sensitivity, loss of pleasure (collectively “arousal disorder”) and/or difficulty in climaxing (“orgasmic disorder”). At the opposite end of the spectrum is premature ejaculation which can severely curtail duration and satisfaction for both participants.

In women, sexual dysfunction can involve lack of threshold desire, loss of sensitivity, loss of pleasure (collectively “sexual arousal disorder”) and/or inability to climax (“orgasmic disorder”). Emotional and psychological rather than physical sexual dysfunction may be more common among women than men. Studies show that women commonly have insecurities about body image and carry stresses and anxieties of life with them into the bedroom. These insecurities and stresses can negatively impact the mood-factor (emotional and psychological state) and inhibit physiological arousal, such as decreased blood flow to the clitoris and labia, often making orgasm unattainable.

While there are pharmaceutical drugs (e.g., sildenafil, tadalafil, vardenafil and avanafil, known as phosphodiesterase type 5 (PDE5) inhibitors) that can remedy the physiological condition of ED and permit men to perform sexually, they generally do not restore lost sensitivity, diminished enjoyment, or difficulty in climaxing. In fact, many men report reduced sensitivity and strength of orgasm when using such drugs. Such drugs are generally ineffective for women because achieving an erection is irrelevant to being able to have sex. PDE5 inhibitors do not adequately address issues involving lack of threshold desire, loss of sensitivity, loss of pleasure, or inability to climax (i.e., because they do virtually nothing to address powerful psychological forces affecting women).

While PDE5 inhibitors can address ED, which permits a man to mechanistically have sexual relations, they do not address other aspects, such as sexual sensitivity and pleasure. In addition, such drugs are currently regulated by the Food and Drug Administration (FDA) and are only available by prescription. They can also cause uncomfortable side effects when taken at full strength, including headache, flushing, nasal congestion, nasopharyngitis, dyspepsia, and prolonged and sometimes painful erections. Accordingly, there remains a long-felt but unmet need to find compositions that effectively and reliably addresses diminished sexual performance and pleasure while reducing the pharmaceutical load of PDE5 inhibitors.

### SUMMARY

The present invention relates to compositions (e.g., pharmaceutical compositions) for increasing sexual pleasure in an adult human in need thereof that have reduced pharmaceutical load of phosphodiesterase type 5 (PDE5) inhibitor. The pharmaceutical preparations include a quantity of a cannabinoid component sufficient to synergistically amplify the effect of the PDE5 inhibitor so that the dosage form of the composition provides no greater than 50% of a full prescription quantity or dose of the PDE5 inhibitor for an adult male while providing at least 80% of the effect of the full prescription quantity or dose for an adult male.

To accomplish these results the compositions (e.g., pharmaceutical compositions) include a combination of: (1) a PDE5 inhibitor that enhances blood flow to the genital region in order to enhance sexual response (e.g., ability to perform and/or time to arousal); and (2) one or more cannabinoid compounds derived from the plant genus Cannabis, which are included in an effective amount and/or in a ratio effective to enhance sexual pleasure (e.g., threshold desire, sensitivity and/or enjoyment) and synergistically amplify the effect of the PDE5 inhibitor. The synergistic combination results in increased ability to perform and enjoyment of intimate sexual activities by men and women, including at least one of increased blood flow to erectile tissue, increased sexual desire, increased sexual arousal, increased sexual sensitivity, or increased strength of orgasm.

In some embodiments, the composition is provided by a dosage form selected from capsules, tablets, oral drops, infused edibles, infused food preparations, infused drink preparations, massage oils, lotions, gels, creams, lubricants, patches, and suppositories.

According to several embodiments, the pharmaceutical preparations can be delivered in a manner so that the time of enhanced sexual response and sexual pleasure coincide or complement each other (i.e., so that both are present at the same time at least some of the time). Methods of delivery include oral delivery, topical delivery, injection, inhalation, or combinations thereof. Advantageously, the components of the pharmaceutical preparations can be delivered together in a single mode of delivery for simplicity and proper dosage (e.g., in a combined oral preparation or a topical preparation). Alternatively, the components of the pharmaceutical preparations can be pre-packaged in a kit and delivered individually, whether simultaneously or sequentially.

### DETAILED DESCRIPTION OF THE PREFERRED EMBODIMENTS

Disclosed herein are compositions (e.g., pharmaceutical compositions) for increasing sexual pleasure in an adult human in need thereof that have reduced pharmaceutical load of phosphodiesterase type 5 (PDE5) inhibitor. The pharmaceutical preparations include a quantity of a cannabinoid component sufficient to synergistically amplify the effect of the PDE5 inhibitor so that the dosage form of the composition provides no greater than 50% of a full prescription quantity or dose of the PDE5 inhibitor for an adult male while providing at least 80% of the effect of the full prescription quantity or dose for an adult male.

The term “sexual pleasure” can include a variety of physiological and/or psychological aspects or conditions that affect the amount of enjoyment of sexual activity. Examples include, but are not limited to, threshold desire to commence sexual activity, physical sensitivity during sexual activity, psychological pleasure or awareness during sexual activity, ability to reach climax, amount of pleasure leading up to climax, quality of climax, duration of climax, and the like.

The term “sexual response” can include a variety of physiological and/or psychological aspects that affect the ability to perform sexual activities. In men, the most common condition is the inability to achieve or maintain an erection. In women, conditions that inhibit sexual response are more varied and complex but include, for example, inability or delay in becoming aroused while being kissed or touched in erogenous zones. In many cases such inability can be more psychological than physiological.

The term “sexual arousal disorder” can apply to men and women and is where a person has difficulty with arousal or are unable to become aroused or maintain arousal during sexual activity.

The term “orgasmic disorder” can apply to men and women and is where a person has persistent or recurrent difficulty in achieving orgasm after sufficient sexual arousal and ongoing stimulation.

The term “low sexual desire” can apply to men and women and is where the person has lack of sexual interest and willingness to be sexual.

According to several embodiments, one or more compounds that enhance sexual pleasure (“pleasure-enhancing component”) include one or more cannabinoid compounds from the plant genus Cannabis. Examples of cannabinoid compounds include tetrahydrocannabinol (“THC”), which is a subgenus of several different isomers having different chiral centers and is the main psychoactive constituent of Cannabis; cannabidiol (“CBD”), which is less or perhaps even non-psychoactive but may modulate certain effects of THC in the nervous system, cannabinol (“CBN”), tetrahydrocannabivarin (“THCV”), cannabigerol (“CBG”), cannabichromene (“CBC”), cannabichromevarin (“CBCV”), cannabidiphorol (“CBDP”), cannabidivarin (“CBDV”), cannabielsoin (“CBE”), cannabigerovarin (“CBGV”), cannabicyclol (“CBL”), cannabinodiol (“CBND”), cannabicitran (“CBTC”), cannabivarin (“CBV”), tetrahydrocannabiorcol (“THCC”), tetrahydrocannabihexol (“THCT”), and tetrahydrocannabiphorol (“THCP”).

Examples of synthesized cannabinoid compounds include dronabinol (Marinol) (a pure isomer of THC, (-)-trans-Δ9-tetrahydrocannabinol, which is the main isomer found in cannabis) and nabilone (a synthetic racemic mixture consisting of the (S,S) and the (R,R) isomers of THC). Synthetic forms of THC and CBD can function the same as plant-based THC and CBD, respectively, and are therefore “cannabinoids” unless expressly excluded.

It was unexpectedly found that combining one or more cannabinoids, particularly THC, but also CBD, CBN, CBG, and other cannabinoids, in sufficient quantities can synergistically amplify the PDE5 inhibiting effect of PDE5 inhibitors so that a same or greater PDE5 inhibiting effect can be attained using a reduced quantity of the PDE5 inhibitor compared the amount required to achieve the same effect in the absence of the one or more cannabinoids.

Without being bound to theory, it is postulated that the one or more cannabinoids, when included in threshold or greater amounts, inhibit enzymatic breakdown of the PDE5 inhibitor and/or act as a contributory PDE5 inhibitor by a same or similar mechanism as the PDE5 inhibitor.

In some embodiments, the quantity of the PDE5 inhibitor (e.g., single PDE5 inhibitor) in the composition is no greater than 50%, or less than about 45%, less than about 40%, less than about 35%, less than about 30%, less than about 25%, or less than about 20%, of a full prescription quantity of the PDE5 inhibitor for an adult male.

In some embodiments, the quantity of the cannabinoid component in the composition is sufficient to amplify the PDE5 inhibiting effect of the PDE5 inhibitor to at least 80%, at least 85%, at least 90%, or at least 95%, of the PDE5 inhibiting effect of the full prescription quantity for an adult male when administered in the reduced quantity.

In some embodiments, the PDE5 inhibitor and the cannabinoid component are effective in increasing sexual pleasure in the adult human in need thereof, including at least one of increased blood flow to erectile tissue, increased sexual desire, increased sexual arousal, increased sexual sensitivity, or increased strength of orgasm in the adult human.

In some embodiments, the PDE5 inhibitor in the composition is sildenafil, which has a full prescription quantity for an adult male of 100 mg such that the composition is preferably formulated and/or provided in a dosage form that provides up to (no more than) 50 mg, up to (no more than) 40 mg, up to (no more than) 30 mg, up to (no more than) 25 mg, or up to (no more than) 20 mg of the sildenafil. Nevertheless, the effect of the sildenafil is amplified the by the cannabinoid component so as to provide at least 80%, at least 85%, at least 90%, or at least 95%, of the full PDE5 inhibiting effect of the full prescription quantity of sildenafil for an adult male.

In some embodiments, the PDE5 inhibitor in the composition is tadalafil, which has a full prescription quantity for an adult male of 20 mg such that the composition is preferably formulated and/or provided in a dosage form that provides up to (no more than) 10 mg, up to (no more than) 8 mg, up to (no more than) 7.5 mg, up to (no more than) 6 mg, or up to (no more than) 5 mg of the tadalafil. Nevertheless, the effect of the tadalafil is amplified the by the cannabinoid component so as to provide at least 80%, at least 85%, at least 90%, or at least 95%, of the full PDE5 inhibiting effect of the full prescription quantity of tadalafil for an adult male.

In some embodiments, the PDE5 inhibitor in the composition is vardenafil, which has a full prescription quantity for an adult male of 20 mg such that the composition is preferably formulated and/or provided in a dosage form that provides up to (no more than) 10 mg, up to (no more than) 8 mg, up to (no more than) 7.5 mg, up to (no more than) 6 mg, or up to (no more than) 5 mg of the vardenafil. Nevertheless, the effect of the vardenafil is amplified the by the cannabinoid component so as to provide at least 80%, at least 85%, at least 90%, or at least 95%, of the full PDE5 inhibiting effect of the full prescription quantity of vardenafil for an adult male.

In some embodiments, the PDE5 inhibitor in the composition is avanafil, which has a full prescription quantity for an adult male of 200 mg such that the composition in preferably formulated and/or provided in a dosage form that provides up to (no more than) 100 mg, up to (no more than) 85 mg, up to (no more than) 70 mg, up to (no more than) 60 mg, or up to (no more than) 50 mg of the avanafil. Nevertheless, the effect of the avanafil is amplified the by the cannabinoid component so as to provide at least 80%, at least 85%, at least 90%, or at least 95%, of the full PDE5 inhibiting effect of the full prescription quantity of avanafil for an adult male.

In some embodiments, the cannabinoid component in the composition comprises tetrahydrocannabinol (THC) and the compositions are preferably formulated and/or provided in a dosage form that contains at least 5 mg, at least 10 mg, at least 15 mg, at least 20 mg, at least 25 mg, or at least 30 mg, of tetrahydrocannabinol (THC).

In some embodiments, the cannabinoid component in the composition comprises cannabidiol (CBD) and the compositions are preferably formulated and/or provided in a dosage form that contains at least at least 10 mg, at least 15 mg, at least 20 mg, at least 25 mg, at least 30 mg, at least 40 mg, or at least 50 mg, of cannabidiol (CBD).

In some embodiments, the cannabinoid component in the composition comprises at least 10 mg, at least 15 mg, at least 20 mg, at least 25 mg, at least 30 mg, or at least 40 mg, of at least one cannabinoid selected from the group consisting of cannabichromene (CBC), cannabichromevarin (CBCV), cannabidiphorol (CBDP), cannabidivarin (CBDV), cannabielsoin (CBE), cannabigerol (CBG), cannabigerovarin (CBGV), cannabicyclol (CBL), cannabinol (CBN), cannabinodiol (CBND), cannabicitran (CBTC), cannabivarin (CBV), tetrahydrocannabiorcol (THCC), tetrahydrocannabihexol (THCH), tetrahydrocannabiphorol (THCP), and tetrahydrocannabivarin (THCV).

In some embodiments, the compositions (e.g., pharmaceutical compositions) are specifically formulated for increasing sexual pleasure in an adult male. In some embodiments, the compositions is in a dosage form that provides a quantity of a single PDE5 inhibitor selected from up to (no more than) 50 mg of sildenafil, up to (no more than) 10 mg of tadalafil, up to (no more than) 10 mg of vardenafil, or up to (no more than) 100 mg of avanafil so to be no greater than 50% of a full prescription quantity of the single PDE5 inhibitor for an adult male, and at least 5 mg, at least 6 mg, at least 8 mg, at least 9 mg, or at least 10 mg, of tetrahydrocannabinol so as to amplify the PDE5 inhibiting effect of the PDE5 inhibitor to at least 80%, at least 85%, at least 90%, or at least 95%, of the PDE5 inhibiting effect of the full prescription quantity for an adult male. The single PDE5 inhibitor and the cannabinoid component are effective in increasing sexual pleasure in the adult male in need thereof, including at least one of increased blood flow to erectile tissue, increased sexual desire, increased sexual arousal, increased sexual sensitivity, or increased strength of orgasm in the adult male.

In some embodiments, the compositions (e.g., pharmaceutical compositions) are specifically formulated for increasing sexual pleasure in an adult female. In some embodiments, the compositions is in a dosage form that provides a quantity of a single PDE5 inhibitor selected from up to (no more than) 40 mg of sildenafil, up to (no more than) 8 mg of tadalafil, up to (no more than) 8 mg of vardenafil, or up to (no more than) 80 mg of avanafil so to be no greater than 40% of a full prescription quantity of the single PDE5 inhibitor for an adult male, and at least 5 mg, at least 6 mg, at least 8 mg, at least 9 mg, or at least 10 mg, of tetrahydrocannabinol so as to amplify the PDE5 inhibiting effect of the PDE5 inhibitor to at least 80%, at least 85%, at least 90%, or at least 95%, of the PDE5 inhibiting effect of the full prescription quantity for an adult male. The single PDE5 inhibitor and the cannabinoid component are effective in increasing sexual pleasure in the adult female in need thereof, including at least one of increased blood flow to erectile tissue (e.g., clitoris and labia), increased sexual desire, increased sexual arousal, increased sexual sensitivity, or increased strength of orgasm in the adult female.

It turns out there are different strains of Cannabis which include differing amounts and/or ratios of cannabinoid compounds. For example, Cannabis sativa typically has a relatively high THC/CBD ratio. Conversely, Cannabis indica has a relatively low THC/CBD ratio compared to Cannabis sativa (although the absolute amount of THC can be higher in Cannabis indica than in Cannabis sativa). There are also several hybrid varieties or strains of Cannabis sativa and Cannabis indica that have intermediate amounts and/or ratios of cannabinoid compounds. The amounts and/or ratios of cannabinoid compounds can change depending on the maturity of the plant, how the plant was grown, amount of artificial or natural light, climate, nutrients, and plant parts being used. In general, the buds and leaves have the highest quantities of cannabinoid compounds, while the stems and seeds have the lowest. In addition, the leaves, stems and seeds can have lower THC/CBD ratio than the buds of the same plant.

Examples of Cannabis sativa dominant strains include Santa Maria, AK-47, Malawi gold, Bazooka, Durban Poison, Maui Waui, Early Bud, Early Pearl, Early Skunk Plant, Great White Shark, Green Spirit, Haze, Haze Skunk, Hempstar, Jack Herer, Kali Mist, Ice, LamsBread x Skunk, Leda Uno, Malawi gold, Niagra x Shiva, Night Queen, Northern Lights x Haze, Power Plant, Purple Haze, Purple Skunk, Smokey Bear, Silver Haze, Shaman, Strawberry Cough, Sweet Island Skunk, Super Silver Haze, Swazi x Skunk, Thai, Voodoo, and White Cloud.

Examples of Cannabis indica dominant strains include Afghani #1, Amstel Gold, Bella Caio, Big Bud, Black Domina, Black African, Black Jack, Chitral, Celtic Cross, Celtic Stone, Chronic, DoubleGum, Early Girl, Early Skunk, Eclipse, Euforia, Green Spirit, G-13, Granddaddy Purple, Hawaiian Skunk, Hindu Kush, Holland's Hope, Hypno, HashPlant, Jack Flash, K2, Lemon Stinky, Mango, Master Kush, Mazar, Mighty Might, Niagra, Northern Lights, Romulan, Pink Indica, Purple High, Purple Urkel, Purple Star, Ruderalis Indica, Shiva, Sour Bubble, Southern Afghani, Super Chrystal, and Twilight.

Examples of more balanced Sativa-indica hybrid strains include Blueberry kush, Rainbow Kashmiri, Blue Velvet, Blueberry, BubbleBerry, Bubblegum, Buddha Plant, Cali Orange Plant, Durban Poison x Mighty Might, Flo, First Mature, Fourway #1, Fruity Pebbles, Full Moon, Jamaican Pearl, Juicy Fruit, GrapeFruit Haze, Himalayan Gold, Island Lady, KC-33, Kerala x Skunk, Kushage, Northern Berry, NYC Diesel, Purple #1, Purple Kush, Romberry, Shiva Shanti, Skunk Red Hair, Skunk Passion, Skunk Haze, Swiss Miss, Turtle Power, and White Widow.

The cannabinoid compounds can be extracted from one or more Cannabis plants using known methods, including organic solvent extraction, water extraction using hot or boiling water, mixed solvents using both an organic solvent and water, heat vaporization, fractional distillation, and the like. Depending on the method of extraction, the identifies and/or ratios of cannabinoid compounds can be altered or selected as desired. In general, extraction is able to provide a better approximation of the actual ratios of cannabinoid compounds found in a particular Cannabis plant as compared to combustion (i.e., smoking). Combustion causes significant destruction of some of the cannabinoid compounds and can change the THC/CBD ratio.

The PDE5 inhibitor enhances sexual response by increasing blood flow to the genital region, typically by increasing the amount of nitric oxide (NO) in the blood, causation dilation of blood vessels. Examples include sildenafil (Viagra®), tadalafil (Cialis®), vardenafil (Levitra®), and avanafil (Stendra®). The cannabinoid component interacts synergistically with the PDE5 inhibitor to amplify its effect so that no more than 50% of the full prescribed amount is required to obtain at least 80% of the benefit provided by the full prescribed amount.

In addition to the PDE5 inhibitor, the compositions may include one or more supplements that can further increase sexual response and/or pleasure. Examples include, but are not limited to, at least 500 mg, 1 g, 1.5 g, 2 g, 2.5 g, 3 g, 4 g, 5 g, or 6 g and up to 20 g, 15 g, 12 g, 10 g, 9 g, or 8 g, or any range between lower and upper values of: L-arginine, L-citrulline, yahimbe root, ginseng (e.g., Korean red ginsing), Ginkgo biloba, horny goat weed, goosefoot, Chenopodium ambrosioides, Chlorophytum borivilianum, Desmodium gangeticum, garlic combined with vitamin C, and/or damiana. Compositions within the scope of the invention may include a pharmaceutically acceptable dose (or dose that is effective to raise blood NO levels) of one or more of the foregoing in order to enhance sexual response and improve performance. A pharmaceutically acceptable (or effective) dose may depend on the gender, weight and/or age of the recipient and will be within known guidelines for these compounds and compositions.

The amount of the foregoing compounds or compositions can vary depending on the potency and mode of action. In general, such compounds or compositions enable men to achieve and maintain an erection by increasing blood flow to the genital region, such as by causing the body to produce nitric oxide. For reasons that may not be well-understood, they also aid women when combined with one or more cannabinoid compounds as disclosed herein, which is surprising and unexpected since they typically have no effect on women when used alone.

The pharmaceutical preparations can have a variety of different modes of delivery, which can be gender-specific or otherwise tailored for the specific needs or desires of the patient. According to an embodiment, the pharmaceutical preparation can be designed as a topical (external or internal, including body cavity, but excluding oral and nasal), e.g., massage oils, lotions, gels, creams, lubricants, genital sprays, vaginal patch, vaginal suppository, or anal suppository. Alternatively or in addition, they can be formulated for ingestion, e.g., capsules, tablets, oral drops, lozenges, lollipops, and food preparations, i.e., “edibles” (aka ingestible, in contrast to sublingual or buccal absorption), such as brownies, cookies, chocolates, chews, gum drops, soft candies, hard candies, liquid shots, and the like). Alternatively, they can be formulated for inhalation into the lungs (e.g., by a heat vaporizer (“vape”) or nebulizer).

Capsules include any delivery form that includes an outer covering enclosing the actives. The outer covering can be any suitable material known in the art, such as gelatin, starch, cellulose ether, gum, protein, or polysaccharide. Tablets include actives compressed into a solid form, sometimes with a binder or inert component. While many capsules and tablets are configured to be swallowed whole, they may also be divided into pieces and swallowed, in some cases chewed and swallowed, sometimes crushed by the teeth to release a liquid, gel or solid that is swallowed. Some tablets or capsules can be used vaginally or anally as suppositories. Or they may be used buccally or sublingually.

A “solid ingestible” includes dosage forms that can be swallowed with no or minimal chewing (e.g., some types of capsules and tablets); dosage forms that are chewed and swallowed, such as food preparations and other edibles (e.g., brownies, cookies, desserts, chocolates, chews, gum drops, soft candies, and some types of capsules and tablets); dosage forms that dissolve in the mouth and are swallowed (e.g., hard candies, lollipops, lozenges, some types of capsules and tablets). A characteristic of a solid ingestible is that the active is intended to be absorbed in the stomach, gut and/or small intestine, as opposed to being primarily absorbed buccally or sublingually.

A “liquid ingestible” includes a liquid or gel that can be swallowed with little or no chewing. A characteristic of a solid ingestible is that the active is intended to be absorbed in the stomach, gut and/or small intestine, as opposed to being primarily absorbed buccally or sublingually. A liquid ingestible can be a shot, a drink, gel pack, oral drops, and the like.

“Dual delivery” compositions can be applied and absorbed in more than one way. Examples include flavored body oils, creams, lotions, liquids, gels, and lubricants, which can be placed on areas of the body where they can be readily absorbed, such as on the skin, especially on or in the genital region, anal region, or armpits of a man or woman, and optionally licked or ingested by the other partner during application and sex play. In some cases, a composition can be placed on or in the genital (or anal) region of one partner and transferred to the genital (or anal) region of the other partner during sex play and intercourse. Such compositions can be placed on or in sex toys, vibrators, dildos, condoms, other prophylactic devices, props, and the like.

In general, extraction of cannabinoids and then delivery without combustion provides superior results compared to smoking weed and ingesting a PDE5 inhibitor. Combustion destroys a significant quantity of cannabinoid compounds and can change their ratios, which makes proper dosing difficult. Smoking weed and ingesting a PDE5 inhibitor also suffers from the inability to control the timing of each, since smoking causes almost instantaneous high while ingesting a PDE5 inhibitor takes time for the body to metabolize. The result can be premature cannabinoid effect, with delayed blood-flow increasing effect coupled with reduced cannabinoid effect when it is desirable for both to be maximized. Delivering the a PDE5 inhibitor and cannabinoid component in a single preparation and/or in the same manner can better control dosing and timing.

Where it is desired to inhale a cannabinoid infused material, such as a liquid, gel or paste, vaporizing apparatus known in the art for delivering nicotine can be used. The concentration of cannabinoids in the vape juice or oil can be adjusted, similar to how it is done when delivering nicotine using a vape stick, hookah, or mod box, so that a predetermined number of puffs will deliver a predetermined amount of the one or more cannabinoids of interest.

Nebulizers known in the art used in hospitals, for hospice or for home care can be used to deliver a predetermined amount of cannabinoids.

In addition to the cannabinoids, the other active for increasing blood flow can be delivered by any suitable means to provide a predetermined quantity of the active. These include oral ingestion, topical delivery, and inhalation, although oral ingestion by capsule or table is currently the most prescribed delivery method.

### EXAMPLES

The following examples demonstrate the synergy when combining a PDE5 inhibitor and THC or other cannabinoid. Male participants rated the strength of their erections and orgasms, and female participants rated the strength of their threshold desire, arousal and orgasms, on a scale of 1 to 10.

### Example 1

A 51 year old male (inventor) consumed ingestible dosage forms of cannabis and sildenafil. This first time, the subject took the full prescription dose of sildenafil (100 mg) in pill form together with an infused cannabis edible containing 20 mg of THC. The subject experienced 5 hours of nearly constant erection, resulting in an uncomfortable episode. What he learned was the amplifying effect of the infused cannabis edible on the PDE5 inhibiting effect of the sildenafil. This was surprising and expected because, when THC is taken alone, it has no noticeable PDE5 inhibiting effect.

The strength of erection was 10, with a duration of 5 hours, which was longer than the warning duration of 4 hours as advertised. Because of the long duration of the effects of sildenafil, the men needed to have sex at least 5 times for pressure relief. The strength of his orgasms was very high, between 8 and 10.

### Example 2

The 51 year old male subject in Example 1 reduced the dosage of sildenafil to 50 mg and used the same cannabis dosage that delivered 20 mg of THC. The subject still experienced 2 hours of nearly constant erection, which was manageable and pleasant. Even at half of the full prescription dosage of sildenafil, because it was combined with 20 mg of THC, his erections were still at a 10, indicating that he experienced 100% of the PDE5 inhibiting effect of sildenafil at half the full prescription dosage. This was surprising and unexpected.

The strength of erection was still 10, with a duration of 2 hours, which was longer than when taking a full 100 mg dosage of sildenafil by itself. Thus, at 50% of the full prescription dosage of sildenafil the subject still obtained 100% of the PDE5 inhibiting effect of sildenafil. The men was able to have sex at least 2 times. The strength of his orgasms was still between 8 and 10 owing to the synergy between the reduced sildenafil dose and the THC.

### Example 3

The 51 year old male subject in Examples 1 and 2 further reduced the dosage of sildenafil to 40 mg, 30 mg, and 20 mg in stages, together with cannabis dosages that delivered 20 mg, 15 mg, or 10 mg of THC. The subject still experienced over 1 hour of erections each time, with a strength of erections at 8, 9 and 10 depending on the dosages of sildenafil and THC. Thus, even when the dosage of sildenafil was reduced to 40%, 30%, and 20% of the full prescription dosage of sildenafil, the subject still obtained 80%, 90% and 100% of the PDE5 inhibiting effect of sildenafil. This was surprising and unexpected.

The strength of erections was between 8 and 10, with a duration of at least 1 hour. The men was able to have sex 1 to 3 times depending on the dosage. The strength of orgasm was between 7 and 10 owing to the synergy between the reduced sildenafil dose and the THC.

### Example 4

A 43 year old male test subject consumed ingestible dosage forms of cannabis and sildenafil. This first time, the subject took 50% of the full prescription dose of sildenafil (50 mg) in pill form together with an infused cannabis edible containing 20 mg of THC. The subject experienced 4 hours of nearly constant erection, resulting in a colossal episode of extended sexual activity with his female sexual partner. The subject also experienced the amplifying effect of the infused cannabis edible on the PDE5 inhibiting effect of the sildenafil. This was surprising and expected because, when the subject took THC alone, it had no noticeable PDE5 inhibiting effect.

The strength of erection was 10, with a duration of 4 hours, which was equal to the warning duration of 4 hours as advertised. Because of the long duration of the effects of sildenafil, the men needed to have sex at least 4 times for pressure relief. The strength of his orgasms was very high, between 8 and 10.

### Example 5

The 43 year old male subject in Example 4 further reduced the dosage of sildenafil to 40 mg and used the same cannabis dosage that delivered 20 mg of THC. The subject still experienced 3 hours of nearly constant erection, which was manageable and pleasant. Even at only 40% of the full prescription dosage of sildenafil, because it was combined with 20 mg of THC, his erections were still at a 10, indicating that he experienced 100% of the full PDE5 inhibiting effect of sildenafil at only 40% of the full prescription dosage. This was surprising and unexpected.

The strength of erection was still 10, with a duration of 3 hours. Thus, at 40% of the full prescription dosage of sildenafil the subject still obtained 100% of the PDE5 inhibiting effect of sildenafil. The men was able to have sex at least 3 times. The strength of his orgasms was still between 8 and 10 owing to the synergy between the reduced sildenafil dose and the THC.

### Example 6

The 43 year old male subject in Examples 4 and 5 further reduced the dosage of sildenafil to 30 mg, 20 mg, and 10 mg in stages, together with cannabis dosages that delivered 20 mg, 15 mg, or 10 mg of THC. The subject still experienced 1 to 2 hours of erections each time, with a strength of erections at 8, 9 and 10 depending on the dosages of sildenafil and THC. Thus, even when the dosage of sildenafil was reduced to 30%, 20%, and 10% of the full prescription dosage of sildenafil, the subject still obtained 80%, 90% and 100% of the PDE5 inhibiting effect otherwise provided by sildenafil at the full prescription dose. This was surprising and unexpected.

The strength of erections was between 8 and 10, with a duration of 1 to 2 hours. The men was able to have sex 1 to 3 times depending on the dosage. The strength of orgasm was between 7 and 10 owing to the synergy between the reduced sildenafil dose and the THC.

### Example 7

A 59 year old male test subject consumed ingestible dosage forms of cannabis and sildenafil. This first time, the subject took 60% of the full prescription dose of sildenafil (60 mg) in pill form together with an infused cannabis edible containing 25 mg of THC. The subject experienced 2 hours of nearly constant erection and had an incredibly strong and powerful sexual episode with his female sexual partner. The subject also experienced the amplifying effect of the infused cannabis edible on the PDE5 inhibiting effect of the sildenafil. This was surprising and expected because, when the subject took THC alone, it had no noticeable PDE5 inhibiting effect.

The strength of erection was 10, with a duration of 2 hours. Because of the long duration of the effects of sildenafil, the men was able to have sex 2 times without losing the erection between episodes. The strength of his orgasms was very high, at 9 and 10.

### Example 8

The 59 year old male test subject in Example 7 further reduced the dosage of sildenafil to 40 mg and used a cannabis dosage that delivered 20 mg of THC. The subject still experienced over 1 hour of nearly constant erection. Even at only 50% of the full prescription dosage of sildenafil, because it was combined with 20 mg of THC, his erections were still at a 10, indicating that he experienced 100% of the PDE5 inhibiting effect of sildenafil at only 50% of the full prescription dosage. This was surprising and unexpected.

The strength of erection was still 10, with a duration of over 1 hour. Thus, at 50% of the full prescription dosage of sildenafil the subject still obtained 100% of the PDE5 inhibiting effect of sildenafil. The men was able to have sex 2 times. The strength of his orgasms was between 9 and 10 owing to the synergy between the reduced sildenafil dose and the THC.

### Example 9

The 59 year old male subject in Examples 7 and 8 further reduced the dosage of sildenafil to 30 mg, 20 mg, and 10 mg in stages, together with cannabis dosages that delivered 20 mg, 15 mg, or 10 mg of THC. The subject still experienced up to 1 hour of erection each time, with a strength of erection at 8, 9 and 10 depending on the dosages of sildenafil and THC. Thus, even when the dosage of sildenafil was reduced to 30%, 20%, and 10% of the full prescription dosage of sildenafil, the subject still obtained 80%, 90% and 100% of the PDE5 inhibiting effect otherwise provided by sildenafil at the full prescription dose. This was surprising and unexpected.

The strength of erections was between 8 and 10, with a duration of up to 1 hour. The men was able to have extended sex at least 1 time depending on the dosage. The strength of orgasm was between 7 and 10 owing to the synergy between the reduced sildenafil dose and the THC.

### Example 10

A 61 year old male test subject suffering from benign prostatic hyperplasia and who had difficulty urinating was prescribed a daily dose of 5 mg of tadalafil, which greatly improved his ability to urinate. The 5 mg dosage of tadalafil was only 25% of the full prescription dosage of tadalafil (20 mg) when prescribed to treat men with severe erectile dysfunction, and only 50% of the reduced prescription dosage of tadalafil (10 mg) when prescribed to treat men with minor erectile dysfunction.

The subject also consumed an infused cannabis edible containing 10 mg, 15 mg, and 20 mg of THC on various occasions. The subject experienced 1 to 2 hours of nearly constant erections and had strong and powerful sexual episodes with his female sexual partner. The subject experienced the amplifying effect of the infused cannabis edible on the PDE5 inhibiting effect of the tadalafil. This was surprising and expected because, when THC was taken alone, it had no noticeable PDE5 inhibiting effect.

The strength of erection was between 9 and 10, with durations of 1 to 2 hours. Because of the long duration of the effects of tadalafil, the men was able to have extended sex 1 to 2 times without losing his erection. The strength of orgasms was between 8 and 10 each time.

### Example 11

The 61 year old male test subject in Example 10 supplemented the low dose of tadalafil with various supplements on nights when sexual intercourse was expected. These included one or more of gingko biloba, Korean red ginseng, horny goat weed, L-arginine, and L-citrulline. The supplements marginally increased the strength and duration of erection and strength of orgasm, even at lower amounts of THC (5 mg, 7.5 mg, and 9 mg). Thus, taking tadalafil, one or more supplements, and 5 to 10 mg of THC had a synergistic effect that greatly enhanced the overall sexual experience. This was surprising and unexpected.

The strength of erection was 10, with a duration of over 1 hour. Thus, at 25% of the full prescription dosage of tadalafil the subject still obtained up to 100% of the PDE5 inhibiting effect of tadalafil. The men was able to have sex 1 to 2 times. The strength of his orgasms was between 8 and 10 owing to the synergy between the reduced tadalafil dose, the supplement(s), and the THC.

### Example 12

The 51 year old male subject in Examples 1 to 3 gave his female sexual partner sildenafil at dosages of 10 mg, 20 mg, 30 mg, and 40 mg, together with THC dosages of 5 mg, 7.5 mg, 10 mg, and 15 mg, in various combinations. The female sexual partner experienced greater initial sexual desire (between 6 and 9) than usual, greater sexual sensitivity (between 6 and 10) than usual, and stronger orgasms (between 7 and 10) than usual. This was surprising and unexpected because sildenafil by itself typically has no sex-enhancing benefits for women. However, the combination the body- and mind-altering effects of the THC and the blood flow enhancing effects of sildenafil to the genital region synergistically interacted to greatly enhance the overall sexual experience.

### Example 13

The 43 year old male subject in Examples 4 to 6 gave his female sexual partner sildenafil at dosages of 10 mg and 20 mg, together with THC dosages of 5 mg, 7.5 mg, and 10 mg in various combinations. The female sexual partner experienced greater initial sexual desire (between 6 and 9) than usual, greater sexual sensitivity (between 6 and 9) than usual, and stronger orgasms (between 7 and 10) than usual. This was surprising and unexpected because sildenafil by itself typically has no sex-enhancing benefits for women. However, the combination the body- and mind-altering effects of the THC and the blood flow enhancing effects of sildenafil to the genital region synergistically interacted to greatly enhance the overall sexual experience.

### Example 14

The 61 year old male subject in Examples 10 to 12 applied a cannabis infused sexual lubricant to his female sexual partner and waited 15 minutes before engaging in sexual relations. The female partner experienced increased clitoral sensitivity than usual, was not intoxicated, and had good orgasms. However, without also taking sildenafil, it is believed she did not obtain the full benefit of greater overall sexual sensitivity, initial arousal, and substantially stronger orgasms.

### Example 15

The 61 year old male subject in Examples 10 to 12 applied a cannabis infused sexual lubricant to his own genitals and waited 15 minutes before engaging in sexual relations. The male subject experienced further increased sexual sensitivity and great orgasms. It is believed that combining the sildenafil, the ingested form of THC, and the cannabis infused sexual lubricant further enhanced the overall benefits of greater sexual sensitivity, powerful erections, and powerful and fully satisfying orgasms.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A composition for increasing sexual pleasure in an adult human in need thereof, comprising:
a dosage form selected from capsules, tablets, oral drops, infused edibles, infused food preparations, infused drink preparations, massage oils, lotions, gels, creams, lubricants, patches, and suppositories, the dosage form containing:
a quantity of a PDE5 inhibitor that is no greater than 50% of a full prescription quantity or dose of the PDE5 inhibitor for an adult male; and
a quantity of a cannabinoid component sufficient to synergistically amplify the PDE5 inhibiting effect of the PDE5 inhibitor to at least 80% of the PDE5 inhibiting effect of the full prescription quantity or dose for an adult male,
wherein the PDE5 inhibitor and the cannabinoid component are effective in increasing sexual pleasure in the adult human in need thereof, including at least one of increased blood flow to erectile tissue, increased sexual desire, increased sexual arousal, increased sexual sensitivity, or increased strength of orgasm in the adult human.

2. The composition of claim 1, wherein the quantity of the PDE5 inhibitor is less than about 45%, less than about 40%, less than about 35%, less than about 30%, less than about 25%, or less than about 20%, of the full prescription quantity or dose for an adult male.

3. The composition of claim 2, wherein the quantity of the cannabinoid component is sufficient to amplify the PDE5 inhibiting effect of the PDE5 inhibitor to at least 85%, at least 90%, or at least 95%, of the PDE5 inhibiting effect of the full prescription quantity or dose for an adult male.

4. The composition of claim 1, wherein the PDE5 inhibitor is sildenafil and the full prescription quantity or dose for an adult male is 100 mg.

5. The composition of claim 4, wherein the dosage form provides up to 50 mg, up to 40 mg, up to 30 mg, up to 25 mg, or up to 20 mg of the sildenafil.

6. The composition of claim 1, wherein the PDE5 inhibitor is tadalafil and the full prescription quantity or dose for an adult male is 20 mg.

7. The composition of claim 6, wherein the dosage form provides up to 10 mg, up to 8 mg, up to 7.5 mg, up to 6 mg, or up to 5 mg of the tadalafil.

8. The composition of claim 1, wherein the PDE5 inhibitor is vardenafil and the full prescription quantity or dose for an adult male is 20 mg.

9. The composition of claim 6, wherein the dosage form provides up to 10 mg, up to 8 mg, up to 7.5 mg, up to 6 mg, or up to 5 mg of the vardenafil.

10. The composition of claim 1, wherein the PDE5 inhibitor is avanafil and the full prescription quantity or dose for an adult male is 200 mg.

11. The composition of claim 6, wherein the dosage form provides up to 100 mg, up to 85 mg, up to 70 mg, up to 60 mg, or up to 50 mg of the avanafil.

12. The composition of claim 1, wherein the cannabinoid component contains at least 5 mg, at least 10 mg, at least 15 mg, at least 20 mg, at least 25 mg, or at least 30 mg, of tetrahydrocannabinol (THC).

13. The composition of claim 1, wherein the cannabinoid component contains at least 10 mg, at least 15 mg, at least 20 mg, at least 25 mg, at least 30 mg, at least 40 mg, or at least 50 mg, of cannabidiol (CBD).

14. The composition of claim 1, wherein the cannabinoid component contains at least 10 mg, at least 15 mg, at least 20 mg, at least 25 mg, at least 30 mg, or at least 40 mg, of at least one cannabinoid selected from the group consisting of cannabichromene (CBC), cannabichromevarin (CBCV), cannabidiphorol (CBDP), cannabidivarin (CBDV), cannabielsoin (CBE), cannabigerol (CBG), cannabigerovarin (CBGV), cannabicyclol (CBL), cannabinol (CBN), cannabinodiol (CBND), cannabicitran (CBTC), cannabivarin (CBV), tetrahydrocannabiorcol (THCC), tetrahydrocannabihexol (THCH), tetrahydrocannabiphorol (THCP), and tetrahydrocannabivarin (THCV).

15. The composition of claim 1, wherein the dosage form comprises a capsule or tablet.

16. The composition of claim 1, wherein the dosage form is selected from massage oil, lotion, gel, cream, lubricant, patch, or suppository.

17. The composition of claim 1, wherein the infused edible, infused food preparation, or infused drink preparation is selected from the group consisting of lozenges, lollipops, brownies, cookies, chocolates, chews, gum drops, soft candies, hard candies, and liquid shots.

18. The composition of claim 1, further comprising a supplement selected from the group consisting of L-arginine, L-citrulline, yahimbe root, ginseng, Korean red ginsing, Ginkgo biloba, horny goat weed, goosefoot, Chenopodium ambrosioides, Chlorophytum borivilianum, Desmodium gangeticum, garlic combined with vitamin C, and/or damiana, and compositions that increase blood flow to the genital region and are not cannabinoids.

19. A composition for increasing sexual pleasure in an adult male in need thereof, comprising:
a dosage form selected from capsules, tablets, oral drops, infused edibles, infused food preparations, infused drink preparations, massage oils, lotions, gels, creams, lubricants, patches, and suppositories, the dosage form containing:
a quantity of a single PDE5 inhibitor selected from up to 50 mg of sildenafil, up to 10 mg of tadalafil, up to 10 mg of vardenafil, or up to 100 mg of avanafil so to be no greater than 50% of a full prescription quantity or dose of the single PDE5 inhibitor for an adult male; and
at least 5 mg, at least 6 mg, at least 8 mg, at least 9 mg, or at least 10 mg, of tetrahydrocannabinol so as to amplify the PDE5 inhibiting effect of the PDE5 inhibitor to at least 80% of the PDE5 inhibiting effect of the full prescription quantity or dose for an adult male,
wherein the single PDE5 inhibitor and the cannabinoid component are effective in increasing sexual pleasure in the adult male in need thereof, including at least one of increased blood flow to erectile tissue, increased sexual desire, increased sexual arousal, increased sexual sensitivity, or increased strength of orgasm in the adult male.

20. A composition for increasing sexual pleasure in an adult female in need thereof, comprising:
a dosage form selected from capsules, tablets, oral drops, infused edibles, infused food preparations, infused drink preparations, massage oils, lotions, gels, creams, lubricants, patches, and suppositories, the dosage form containing:
a quantity of a single PDE5 inhibitor selected from up to 40 mg of sildenafil, up to 8 mg of tadalafil, up to 8 mg of vardenafil, or up to 80 mg of avanafil so to be no greater than 40% of a full prescription quantity of the single PDE5 inhibitor for an adult male; and
at least 5 mg, at least 6 mg, at least 8 mg, at least 9 mg, or at least 10 mg, of tetrahydrocannabinol so as to amplify the PDE5 inhibiting effect of the PDE5 inhibitor to at least 80% of the PDE5 inhibiting effect of the full prescription quantity or dose for an adult male,
wherein the single PDE5 inhibitor and the cannabinoid component are effective in increasing sexual pleasure in the adult female in need thereof, including at least one of increased blood flow to erectile tissue, increased sexual desire, increased sexual arousal, increased sexual sensitivity, or increased strength of orgasm in the adult female.
